(19) United States
(12) Patent Application Publication
(10) Pub. No.: US 2023/0218382 A1
(43) Pub. Date: Jul. 13, 2023

(54) DEVICE TO AID PREPARATION OF GRAFT FOR DESCEMENT'S MEMBRANE ENDOTHELIAL KERATOPLASTY

(71) Applicant: Summit Medical, LLC, St. Paul, MN (US)

(72) Inventors: Rajesh FOGLA, Hyderabad (IN); Kevin D. MCINTOSH, Brooklyn Park, MN (US); Matthew FRANKS, Bristol (GB)

(73) Assignee: Summit Medical, LLC, St. Paul, MN (US)

(21) Appl. No.: 18/054,663

(22) Filed: Nov. 11, 2022

Related U.S. Application Data
(60) Provisional application No. 63/278,348, filed on Nov. 11, 2021.

(51) Int. Cl. A61F 2/00; A61F 2/14

(52) U.S. Cl. CPC A61F 2/0095; A61F 2/142; A61F 2240/001

(57) ABSTRACT

A device that facilitates preparation of corneal grafts, including an illuminated base; and a corneal graft holder that can be selectively coupled to the illuminated base to illuminate a portion of donor cornea to facilitate preparation of the corneal graft.

### RELATED APPLICATION

This application claims the benefit of U.S. Provisional Patent Application No. 63/278,348, filed on Nov. 11, 2021, the entirety of which is incorporated herein by reference.

### TECHNICAL FIELD

Embodiments of the disclosure relate to corneal surgery. More particularly, embodiments of the disclosure relate to the preparation of donor tissue for corneal transplant. Even more particularly, embodiments of the disclosure relate to preparation of donor material to be harvested for Descemet's membrane (DM) and corneal endothelium to be transplanted in Descemet's Membrane Endothelial Keratoplasty.

### BACKGROUND

Corneal transplants have been performed for the treatment of various corneal disease, injury and dystrophy for decades. Full thickness corneal transplants involve the removal and replacement of a centrally located plug of the cornea including all of its layers. These layers from anterior to posterior include the corneal epithelium, Bowman's membrane, the corneal stroma, Descemet's membrane and the corneal endothelium. Partial thickness corneal transplants are also performed. Partial thickness corneal transplants involve transplantation of less than all of the layers of the cornea.

One form of partial thickness corneal transplant is Descemet's Membrane Endothelial Keratoplasty (DMEK). This procedure is commonly used to treat corneal disease or dystrophy in which the corneal endothelium is diseased, damaged or dystrophic. One common condition treated in this way is Fuch's corneal dystrophy.

DMEK involves the harvesting of Descemet's membrane and its associated corneal endothelium tissue from a donor cornea. The harvested DMEK graft includes the two innermost layers of the cornea which are Descemet's membrane and the corneal endothelium. The average combined thickness of these structures is approximately 14 to 20 μm. The corneal endothelium, in particular, is a delicate tissue. Preservation of the integrity of the corneal endothelium is critical during donor preparation. Descemet's membrane is the basement membrane of the corneal endothelium and is dissected away from the corneal stroma to separate it from the corneal stroma during the preparation procedure.

A number of graft harvesting techniques have been described for DMEK donor tissue preparation. These include variations of manual dissection, pneumatic dissection and hydro dissection. All of these techniques are generally performed under illuminated magnification using operating microscopes similar to those used during the surgical procedure itself. Clinically, the reported rate of tissue loss for preparation of DMEK grafts can range from 2% to 20% the risk of tearing or otherwise damaging the DMEK graft during preparation is significant and is higher for less experienced surgeons and for those initially learning the procedure. In general, there is a shortage of donor corneas available in the world. Therefore any donor corneas lost in tissue preparation is a serious problem.

During the DMEK procedure a small incision is made in the peripheral cornea and the DMEK graft is inserted through a tubular inserter in a rolled up state. The graft is then carefully unrolled and placed in contact with the existing damaged corneal endothelium. It is important that the Descemet's membrane of the graft be placed in contact with the existing endothelium. Accordingly the grafts are marked so that the surgeon can readily determine whether the graft is properly oriented. Typically, a gas bubble is placed in the anterior chamber of the eye to assist in holding the graft in place. The patient is then required to lay on their back for a period of time to facilitate acceptance of the graft.

DMEK is currently the preferred technique for endothelial keratoplasty. It provides better visual outcomes, faster visual rehabilitation and a lower risk of graft rejection as compared to Descemet's stripping automated endothelial keratoplasty (DSAEK). However in DMEK, donor graft preparation involves manual stripping of Descemet's membrane from the donor cornea. This is in contrast to DSAEK wherein an automated microkeratome is used to prepare the donor lenticule. Donor grafts for DMEK are typically prepared by the surgeon immediately prior to surgery or one day in advance of surgery. Such grafts may also be obtained as pre-stripped corneal tissue from an eye bank. Due to the steep learning curve involved in donor cornea preparation, most DMEK surgeons rely on pre-stripped donor tissue from eye banks. The availability of pre-stripped tissue is not uniform worldwide and many surgeons without access to eye banks have no other choice than to learn to strip Descemet's membrane from donor cornea if the procedure is to be performed. In addition, the manual stripping procedure has the highest success rate with least damage to donor corneal endothelium. Manual stripping involves creating an initial peripheral break followed by peeling of the Descemet's membrane using forceps or other instruments. Healthy endothelium and Descemet's membrane are transparent as is the adjacent corneal stroma. Even under an operating microscope and associated illumination it can be difficult for the surgeon to visualize the graft during the stripping procedure.

Another important concern in the preparation of donor grafts for DMEK is maintenance of sterility of the tissue to be transplanted. Because of the nature of DMEK, a partial thickness of donor tissue is inserted into the anterior chamber within the globe of the eye. Eye surgeons are well aware of the concern of introducing pathogens into the globe which can possibly lead to the development of endophthalmitis, an infection within the eye that can be very difficult to treat and which can be sight threatening. Thus, maintaining the sterility of the donor tissue that is used to prepare the graft is important.

Accordingly, there is still room for improvement in techniques related to the preparation of such DMEK grafts.

### SUMMARY

Embodiments of the disclosure address many of the above problems and provide a benefit in the field of DMEK draft preparation.

In one embodiment, a device that facilitates preparation of a corneal graft comprises an illuminated base; and a corneal graft holder that can be selectively coupled to the illuminated base to illuminate a portion of a donor cornea to facilitate preparation of the corneal graft.

In another embodiment, a method comprises providing a graft preparation device comprising an illuminated base and a corneal graft holder, wherein corneal graft holder is arranged to receive a donor cornea and the illuminated base is arranged to illuminate the donor cornea.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of a device for assisting DMEK corneal graft processing according to an example embodiment of the disclosure.

FIG. 2 is a perspective view of the device of FIG. 1 and bowl donor material support structure according to an example embodiment of the disclosure.

FIG. 3 is a perspective view of the bowl donor material support structure in position on the device according to FIG. 1.

FIG. 4 is a schematic depiction of several different illumination patterns utilized in a ring light according to an example embodiment of the disclosure.

FIG. 5 is circuitry schematic according to an example embodiment of the disclosure.

FIG. 6 is a cross sectional schematic depiction of a reusable illumination base in combination with a disposable pre-sterilized graft holder with integral sterile drape according to an example embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

An example embodiment of the disclosure includes an illuminated DMEK donor tissue support that illuminates the DMEK graft during the preparation and stripping of Descemet's membrane. Because the graft is illuminated in such a way that it highlights areas of importance during surgery, embodiments of the disclosure facilitate dissection of Descemet's membrane from the corneal stroma. As discussed above, Descemet's membrane and the endothelium are very fragile and can be easily torn during the stripping process. In addition, even under a surgical microscope with attendant surgical illumination it can be difficult for the surgeon to visualize where the edges of the tissue lie and what areas of Descemet's membrane are still attached to the corneal stroma. The illuminated DMEK donor tissue support according to an example embodiment of the disclosure allows the surgeon to work on the graft while the graft is illuminated in such a way to highlight the edges of the tissue and the areas where Descemet's membrane is still adherent to the corneal stroma.

An example embodiment can include one or more of a number of features: a ring light of, for example, LEDs, positioned to illuminate the graft from around and below the graft. The LEDs may include white LEDs or other colored LEDs such as, for example, blue or red LEDs. The illuminated DMEK donor tissue support may include a black background or another color background to provide contrasting background behind the illuminated graft. According to some example embodiments of the disclosure, the ring light is adjustable in brightness. The ring light may include selectable patterns of illumination for LEDs or other sources of illumination such that only a portion of the ring may be illuminated at a given time or the entire ring may be illuminated.

Example embodiments may utilize vacuum to secure the donor tissue in a manner similar to a DMEK vacuum punch known to those skilled in the art. A supporting member may include a molded transparent bowl, translucent bowl or colored bowl to facilitate light passage. According to example embodiments of the disclosure, the DMEK donor tissue support may be adjustable in height to be at a height comfortable for the surgeon. Example embodiments of the disclosure include cutouts on the sides that facilitate easy access to the bowl by the surgeon.

An example embodiment may include controls including an on/off switch, a variable brightness adjuster and a pattern change selector. According to an example embodiment of the disclosure, patterns may include illumination of the entire ring, illumination of a single light source for example at 3 o'clock, illumination of two opposing light sources for example at 3 o'clock and 9 o'clock, illumination of 3 light sources equally spaced about the circle, illumination of two adjacent light sources, illumination of two sets of two adjacent light sources oriented in opposition to one another and illumination of three adjacent light sources illuminating 180° of a circle.

According to an example embodiment, the supporting well for the donor tissue is watertight in view of the fact that is often desirable to apply liquids such as balanced salt solution (BSS) or a dye to the donor tissue. According to an example embodiment, the device may be adapted or configured to also be utilized during the trephination process along with a corneal trephine.

Additional embodiments of the disclosure may include a method of preparing a donor graft utilizing posterior illumination of the donor graft.

Another additional embodiment may include application of a ring light array to illuminate a donor graft during processing.

A further additional embodiment may include an illuminated corneal DMEK trephination device.

A further additional embodiment may include an illumination station also having a suction option for securing the donor corneal tissue.

Another example embodiment may include a modular instrument that allows conversion to a trephination station in addition to the corneal graft processing station.

A further example embodiment may utilize the light array at a selected diffraction angle. In addition, light polarization in the illumination system may facilitate detection of defects in the harvested tissue. A further example embodiment of the disclosure includes a bowl donor material support structure made of Makrolon in a variety of colors to facilitate easier viewing.

According to another example embodiment of the disclosure, the illumination base is a reusable structure and the DMEK graft holder is a pre-sterilized disposable structure that incorporates an integral sterile drape that covers at least the reusable illumination base. In a further example embodiment, the DMEK graft holder is a pre-sterilized disposable structure that incorporates an integral sterile drape that covers the reusable illumination base as well as a substantial area of work surface surrounding the illumination base.

According to example embodiments of the disclosure, the integral sterile drape may cover a work surface surrounding the DMEK graft holder over a radius of, for example, between 15 cm and 50 cm. While this distance is described as a radius there is no need for the integral sterile drape to be circular. The drape structure may take on any convenient perimeter shape.

According to another example embodiment of the disclosure the integral sterile drape may be a rigid or semi rigid structure formed to conform to the shape of the reusable illumination base and also extending peripherally outwardly beyond the reusable illumination base. According to this example embodiment of the disclosure, the pre-sterilized disposable graft holder and sterile drape facilitates preparation by a surgical technician who can conveniently arrange the structures prior to the surgeon using them.

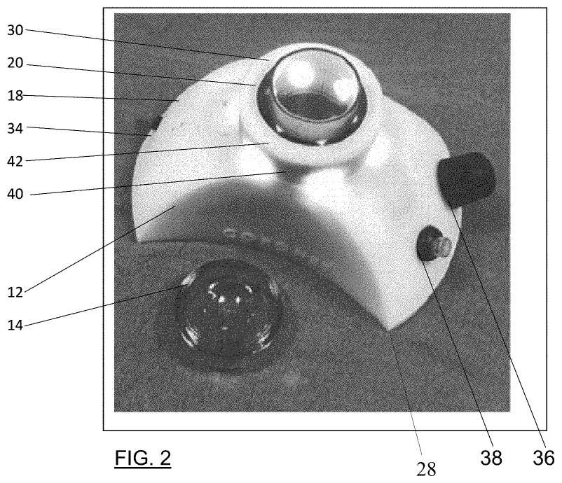

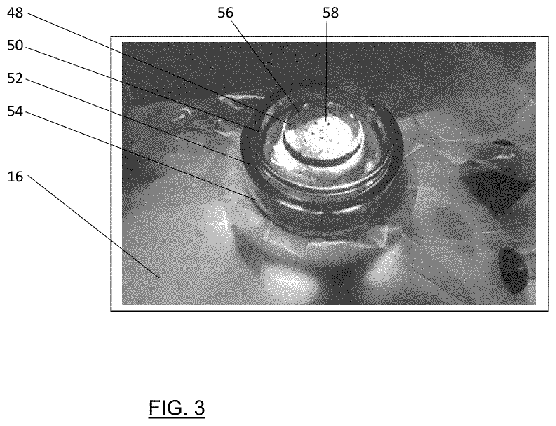

Referring to FIGS. 2 and 3, graft preparation device 10 generally includes illumination base 12 and DMEK graft holder 14. FIG. 2 depicts DMEK graft holder 14 adjacent to and separated from illumination base 12. FIG. 3 depicts DMEK graft holder 14 in place for use on illumination base 12. In addition, in FIG. 3 sterile drape 16 is depicted.

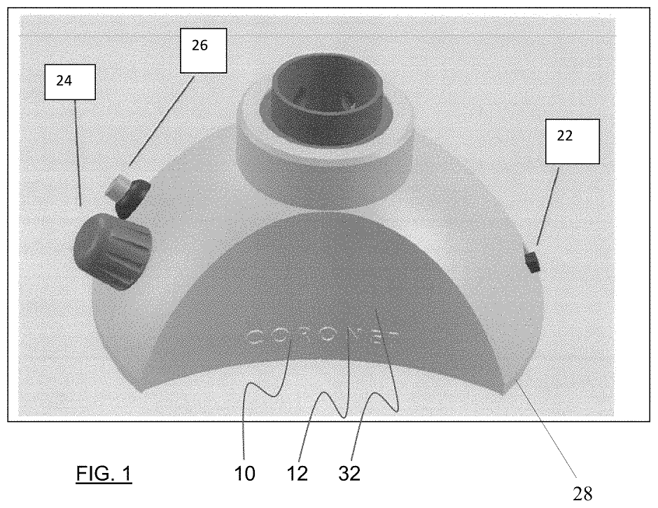

Referring to FIGS. 1, 2 and 3, illumination base 12 generally includes housing 18, holder port 20, switch 22, brightness adjuster 24 and pattern selector 26.

Housing 18, in the depicted embodiment, includes generally hemispherical portion 28 and cylindrical port support 30. Hemispherical portion 28 presents access cutouts 32. Access cutouts 32 provide space for the hands of the surgeon for ease of access. Hemispherical portion 28 further presents switch opening 34, brightness adjuster opening 36 and pattern selector opening 38.

Cylindrical port support 30, in the depicted embodiment, is integrally formed with hemispherical portion 28. This should not be considered limiting. Cylindrical port support 30 extends upwardly from hemispherical portion 28. Cylindrical port support 30 includes cylindrical portion 40 and rim portion 42. In the depicted embodiment cylindrical port support 30 encloses ring light 44 including for example six LEDs 46. The number of LEDs is an example and should not be considered limiting.

Holder port 20 is positioned inside cylindrical port support 30. Holder report 20 can be formed from a transparent or translucent material and is generally cylindrical in shape. Holder port 20 is located within ring light 44 in the depicted embodiment. Holder port 20 is sized and shaped to receive DMEK graft holder 14 thereon or therein. Holder port 20 extends upwardly beyond cylindrical port support 30 and facilitates coupling of DMEK graft holder 14 to illumination base 12.

Switch 22 is adapted to fit switch opening 34 and is operably coupled to ring light 44. Switch 22 controls the illumination of ring light 44 and is adapted to switch ring light 44 on and off.

Brightness adjuster 24 is adapted to fit brightness adjuster opening 36. Brightness adjuster 24 is operably coupled to ring light 44 and also to switch 22. Brightness adjuster 24 is adapted to control the illumination level of ring light 44.

Pattern selector 26 is adapted to fit pattern selector opening 38. Pattern selector 26 is operably coupled to ring light 44 and to circuit board (not shown). Pattern selector 26 is adapted to control and select illumination patterns of LEDs 46 of ring light 44.

DMEK graft holder 14, according to an example embodiment, is a unitary structure formed of a transparent or translucent material. DMEK graft holder 14 generally includes raised bowl portion 48, plateau portion 50, rim portion 52 and cylindrical portion 54.

Raised bowl portion 48 presents concave graft receiver 56. Concave graft receiver 56 may be pierced by vacuum holes 58.

Plateau portion 50 generally surrounds raised bowl portion 48 and, according to an example embodiment, may be generally planar. Plateau portion 50 is bounded by circular rim 60 which terminates at rim portion 52.

Rim portion 52 exists that the juncture between plateau portion 50 and cylindrical portion 54. Cylindrical portion 54 extends downwardly from rim portion 52 and is sized to fit over and to be received over holder port 20 in this example embodiment.

According to an example embodiment, DMEK graft holder 14 may be formed from Makrolon material by molding. For example, DMEK graft holder 14 may be formed by injection molding.

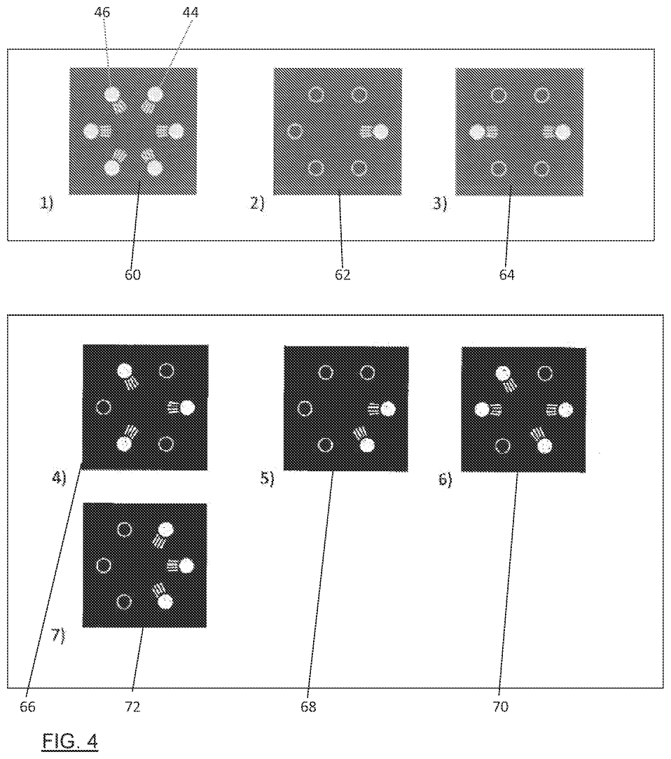

Referring now to FIG. 4, ring light 44 maybe formed by the positioning of LEDs 46. LEDs 46 may be controlled by pattern selector 26 to be illuminated in a variety of patterns. In FIG. 4, seven patterns are depicted. The depicted patterns are presented as examples and should not be considered limiting.

Example ring light patterns include pattern one 60, pattern two 62, pattern three 64, pattern four 66, pattern five 68, pattern six 70 and pattern seven 72.

In pattern one 60, all six LEDs 46 are illuminated. In pattern two 62, a single LED 46 is illuminated. In pattern three 64, two diametrically opposed LEDs 46 are illuminated. In pattern four 66, three LEDs equally spaced are illuminated. In pattern five 68, two adjacent LEDs are illuminated. In pattern six 70, two sets of two adjacent LEDs are illuminated. In pattern seven 72, three adjacent LEDs are illuminated. LEDs 46 may emit white light or any other color light in order to facilitate viewing. LEDs 46 may all be of a single wavelength or may be of mixed wavelengths.

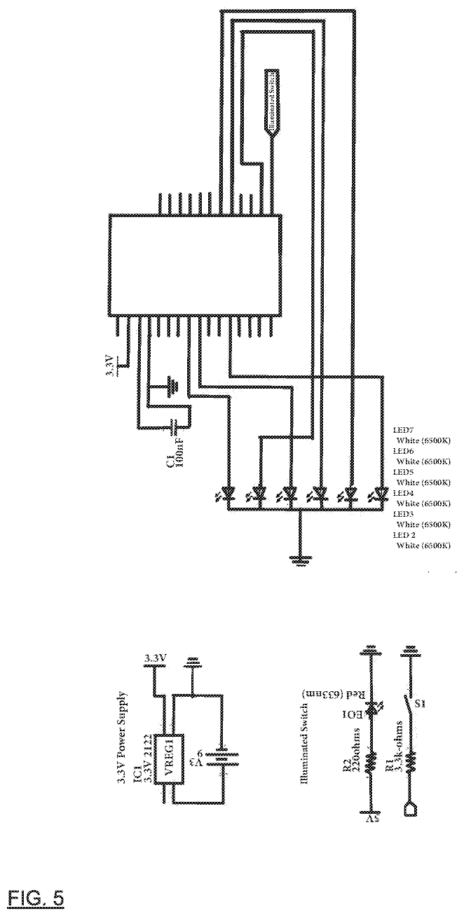

Referring now to FIG. 5, an example electrical schematic is depicted. Other electrical schemes are available to operate ring light 44 as is known to those skilled in the art.

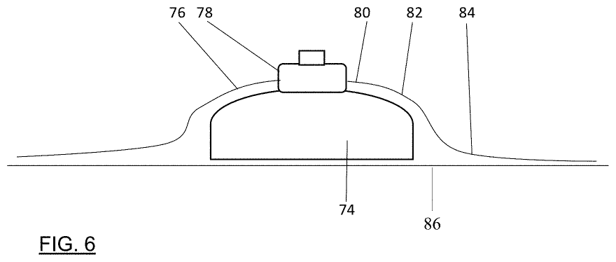

Referring to FIG. 6, a cross-sectional schematic view of reusable illumination base 74 along with a disposable sterile combination graft holder/integral sterile drape 76 is depicted.

Reusable illumination base 74 is generally similar in structure and design to illumination based 12 described above. Accordingly, the general structure of reusable illumination base 74 will not be further described here.

To accommodate use with graft holder/integral sterile drape 76, switch 22, brightness adjuster 24 and pattern selector 26 may be, for example, remotely located from the rest of reusable illumination base 74. Switch 22, brightness adjuster 24 and pattern selector 26 may be operably coupled to reusable illumination base 74 by wired or wireless connection. For example, Bluetooth connection may be utilized. Otherwise, the structure and function of reusable illumination base 74 is similar to illumination base 12.

In the depicted embodiment graft holder/integral sterile drape 76 generally includes DMEK graft holder portion 78 and integral sterile drape portion 80. Graft holder portion 78 may be similar in structure to graft holder 14 described above.

Sterile drape portion 80 generally includes illumination base portion 82 and work surface portion 84. According to an example embodiment, sterile drape portion 80 may be formed of a pliable liquid impervious material that is sufficiently pliable to drape over both illumination base portion 82 and an underlying work surface.

According to another example embodiment, sterile drape portion 80 may be formed of a more rigid or semirigid impervious material that is shaped so that illumination base portion 82 conforms generally to the shape of reusable illumination base 74 and work surface portion 84 extends outwardly away from illumination base portion 82 in a generally planar fashion. Further, illumination base portion 82 can be formed of a material of differing rigidity from work surface portion 84. For example, illumination base portion 82 can be formed of a material of greater rigidity than work surface portion 84 to facilitate work surface portion 84 conforming to a work surface of irregular shape.

According to yet another example embodiment, sterile drape portion 80 may be formed of an absorbent material adapted to capture liquid that might be utilized or released in the graft preparation procedure.

In operation, a surgeon or technician places graft preparation device 10 on a work surface at a convenient height. Sterile drapes 16 may be placed over illumination base 12 of graft preparation device 10 prior to placing DMEK graft holder 14 on illumination base. The surgeon or technician activates switch 22 to illuminate ring light 44. The surgeon or technician manipulates brightness adjuster 24 and pattern selector 26 to select a desired brightness and pattern.

Illumination base 12 is positioned so that access cutouts 32 are located roughly at three o'clock and nine o'clock relative to the surgeon or technician. This orientation permits maximum space for the surgeon's or technician's hands proximate illumination base 12.

A harvested donated cornea is placed on concave graft receiver 56 endothelium side up. In embodiments where vacuum is applied to vacuum holes 58, vacuum may be applied to secure donated corneal tissue. This can occur prior to or following placement of DMEK graft holder 14 on illumination base 12. The surgeon or technician can manipulate brightness adjuster 24 and pattern selector 26 as desired to facilitate best viewing of Descemet's membrane and the endothelium relative to the corneal stroma. The surgeon or technician may select any one of pattern one 60, pattern two 62, pattern three 64, pattern four 66, pattern five 68, pattern six 70 or pattern seven 72 and a desired brightness level.

The surgeon or technician then proceeds to separate Descemet's membrane from the corneal stroma by techniques known to those of skill in the art to prepare the DMEK graft.

In the case of reusable illumination base 74 and graft holder/integral sterile drape 76, the surgeon or the technician places reusable illumination base 74 on a work surface and applies graft holder/integral sterile drape 76 over reusable illumination base 74 and at least a part of the work surface.

The surgeon or technician then proceeds to separate Descemet's membrane from the corneal stroma by techniques known to those of skill in the art to prepare the DMEK graft similar to with other embodiments.

Upon completion of graft preparation graft holder/integral sterile drape 76 may be removed and properly discarded. Then a further graft holder/integral sterile drape 76 may be applied to facilitate a further graft preparation procedure. This approach provides simplicity while facilitating maintenance of the sterile field for graft preparation.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A device that facilitates preparation of a corneal graft, comprising:
an illuminated base; and
a corneal graft holder that can be selectively coupled to the illuminated base to illuminate a portion of a donor cornea to facilitate preparation of the corneal graft.

2. The device of claim 1, wherein the illuminated base is arranged to illuminate the corneal graft to highlight the edges of the corneal graft and a Descemet's membrane.

3. The device of claim 1, wherein the illuminated base illuminates the corneal graft from around and below the corneal graft.

4. The device of claim 1, wherein the illuminated base comprises one or more light emitting diodes (LEDs).

5. The device of claim 4, wherein each of the LEDs can be selectively illuminated independently of the other LEDs.

6. The device of claim 4, wherein the illuminated base comprises a plurality of LEDs are arranged in a circular shape.

7. The device of claim 1, further comprising a supporting member arranged adjacent to a bowl.

8. The device of claim 7, wherein the bowl is at least one of molded, transparent, translucent, colored, or watertight.

9. The device of claim 1, further comprising an illuminated corneal Descemet's Membrane Endothelial Keratoplasty (DMEK) trephination device.

10. The device of claim 1, wherein the corneal graft holder is a pre-sterilized disposable structure.

11. The device of claim 10, wherein the corneal graft holder incorporates an integral sterile drape that covers the illumination base.

12. The device of claim 11, wherein the integral sterile drape is a rigid or semi rigid structure formed to conform to the shape of the illumination base.

17. A method comprising:
providing a graft preparation device comprising an illuminated base and a corneal graft holder,
wherein corneal graft holder is arranged to receive a donor cornea and the illuminated base is arranged to illuminate the donor cornea.
